(19) United States
(12) Patent Application Publication
(10) Pub. No.: US 2025/0010054 A1
(43) Pub. Date: Jan. 9, 2025

(54) CLOSURE CAP FOR LIQUID CONNECTIONS OF A DIALYSER

(71) Applicant: FRESENIUS MEDICAL CARE DEUTSCHLAND GMBH, Bad Homburg (DE)

(72) Inventors: Sufian HASAN, Bad Homburg (DE); Lukas UTZIG, Bad Homburg (DE)

(73) Assignee: FRESENIUS MEDICAL CARE DEUTSCHLAND GMBH, Bad Homburg (DE)

(21) Appl. No.: 18/712,256

(22) PCT Filed: Nov. 28, 2022
(86) PCT No.: PCT/EP2022/083461

§ 371 Date: May 22, 2024

(30) Foreign Application Priority Data
Nov. 29, 2021 (DE) 10 2021 131 267.1

(51) Int. Cl. A61M 39/10; A61M 1/14

(52) U.S. Cl. CPC A61M 39/10; A61M 1/14

(57) ABSTRACT

The invention relates to a closure cap for closing two different fluid ports of a dialyzer, the closure cap having a first receiving portion for receiving a first fluid port and a second receiving portion for receiving a second fluid port of a dialyzer. Further, the invention relates to a dialyzer wherein two first fluid ports for blood are each closed with the first receiving portion of a closure cap or at least one second fluid port for dialysate or filtrate is closed with the second receiving portion of a closure cap. Furthermore, the invention relates to a closure cap holder for receiving a closure cap according to the invention via the first or the second receiving portion.

The present application relates to a closure cap for closing fluid ports on a dialyzer.

Dialyzers are hollow fiber membrane filters used in extracorporeal blood treatment of patients in various therapies. A basic prerequisite for the use of dialyzers is that they are sterile and free from particles. To ensure the sterility of dialyzers, heat sterilization procedures or sterilization procedures by irradiation are used. In addition to sterility, at least the blood contact surfaces of the dialyzer must also be free from particles. Contamination of a dialyzer with particles can lead to dangerous exposure of a patient. To ensure low particle contamination of a dialyzer when delivered, the fluid ports of the dialyzer are sealed with closure caps after rinsing and sterilization during their production. In particular, the blood ports of the dialyzers are regularly sealed with caps to keep the blood side of the dialyzers free of particles. Depending on the type of dialyzer, it may also be necessary to keep the dialysate side of a dialyzer free of particles. It is therefore advantageous to also close the dialysate ports with caps and to protect the interior of the hollow fiber membrane filters from particle contamination.

In addition, closure caps for the fluid ports of a dialyzer are also useful for closing the dialyzer after a blood treatment has been performed. After an extracorporeal blood treatment, the dialyzer may still be partially filled with treatment fluid and/or blood. In particular for reasons of hygiene, care must be taken to ensure that the area surrounding a patient's treatment station is not contaminated with residual treatment fluids and/or blood from the patient when dismantling the dialysis machine and the treatment device.

Closure caps are provided in high quantity according to the demand of dialyzers. It is therefore desirable to manufacture closure caps in a cost-effective way. Previous developments have been directed towards providing closure caps that can close both a first fluid port and a second fluid port of a dialyzer. The dialyzers usually have two fluid ports for blood. Furthermore, such dialyzers have at least one fluid port for a treatment fluid or for filtrate. Dialyzers addressed in this application usually have two fluid ports for blood and two fluid ports for a treatment fluid, e.g. a dialysis fluid. The geometrical design of the fluid ports of a dialyzer are specified by standards, e.g. as described in DIN EN ISO 8637: March 2014. However, depending on the type of therapeutic procedure or the geographical region, not all dialyzers have to be designed according to a corresponding standardization.

U.S. Pat. No. 5,954,957 discloses a closure cap for closing a blood port and a dialysate port on a dialyzer. The closure cap shown in U.S. Pat. No. 5,954,957 has a first receiving portion that is used to close the dialysate port. A second receiving portion of the closure cap is used for closing a blood port.

EP 3 765 114 B1 discloses a closure cap having a first cylindrical sidewall and a second cylindrical sidewall, wherein the first cylindrical sidewall is adapted to cooperate with and close the male cone portion of a blood port of a dialyzer. The second side wall of the closure cap surrounds the first side wall so that a channel is formed between the first side wall and the second side wall which is geometrically designed to cooperate with and close a dialysate port of the dialyzer.

### Object of the Invention

Based on the closure caps described in the prior art, there is still a need to develop alternative closure caps that are optimized in terms of manufacturing, handling and functionality.

### SUMMARY OF THE INVENTION

In a first aspect of the invention, the solution to the afore-mentioned problem is provided by the features of claim 1. Dependent claims 2 to 17 represent preferred embodiments.

In a second aspect of the invention, the problem is solved by a dialyzer according to claims 18 and 19.

In a third aspect of the invention, the problem is solved by a cap holder according to claims 20 and 21.

In a fourth aspect of the invention, the problem is solved by a dialysis machine according to claim 22.

### DESCRIPTION

The closure cap described according to the first aspect is suitable for closing the fluid ports of a dialyzer to ensure a sterile and particle-free dialyzer, in particular, the closure cap is configured to cooperate with a first fluid port of a dialyzer in a liquid-tight manner. Provided that the connection between the closure cap and fluid ports of a dialyzer is liquid-tight, it can also be assumed that the connection protects against contamination and ensures sterility and absence of particles. In particular, the closure cap is configured to cooperate in a liquid-tight manner with the blood port of a dialyzer for extracorporeal blood treatment. In addition, the closure cap is also configured to cooperate with a second fluid port of a dialyzer, the geometric configuration of which is different from the first fluid port, so that sterility of the dialyzer and absence of particles can be ensured. In particular, the closure cap is configured such to cooperate with the dialysate port of a dialyzer in a liquid-tight manner. It is possible with the closure cap according to the invention to close two different fluid ports of a dialyzer with one type of closure cap. It is therefore not necessary to produce 2 different caps. Manufacturing effort for producing closure caps according to the invention is therefore significantly reduced.

Advantageously, the closure caps are already attached to the fluid ports directly after the dialyzer has been sterilized and the dialyzer is delivered in this configuration, thus ensuring sterility and a particle-free dialyzer. It is common practice to supply dialyzers in sterile outer packaging and only the blood ports are then sealed with caps. The closure caps according to the invention have the advantage that they can be reused after extracorporeal blood treatment of a patient, for example, for closing the fluid ports of the dialysate. It is therefore not necessary to provide separate closure caps for the dialysate ports.

The handling of the closure caps during therapeutic procedures is therefore simplified. When using the closure caps according to the invention, these are removed immediately before an extracorporeal blood treatment from the dialyzer and can be fixed to a provided closure cap holder according to the invention. Commonly, after a blood treatment session of a patient, it is then necessary to close the dialysate ports of the dialyzer to prevent residual dialysis fluid from leaking out of the dialysate side of the dialyzer. For this purpose, the closure caps attached to the sealing cap holder are used, which previously held the dialyzer's blood port closed when the dialyzer was delivered. The cap holder described in accordance with the present invention thus enables free handling of the closure caps according to the invention in the course of setting up and taking down a dialysis machine in the course of extracorporeal blood treatment. It is in particular advantageous to attach the closure cap holder directly to a dialysis machine so that the staff has the opportunity of placing the closure caps near the used dialyzer.

Further details, features and advantages of the invention become apparent from the following description and drawings. It is shown in:

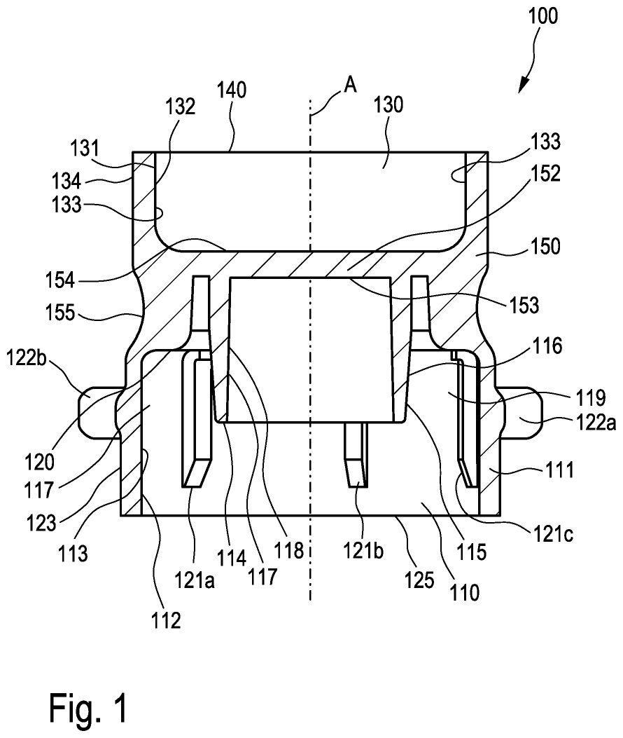

FIG. 1, a sectional drawing of a closure cap according to the invention,

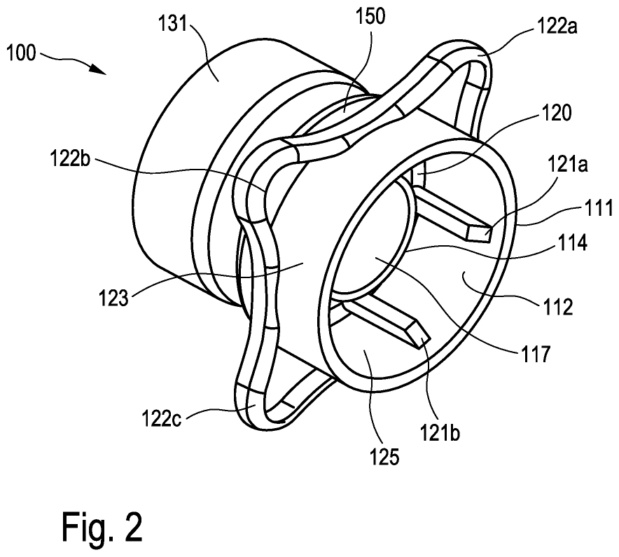

FIG. 2, a side view of a closure cap according to the invention,

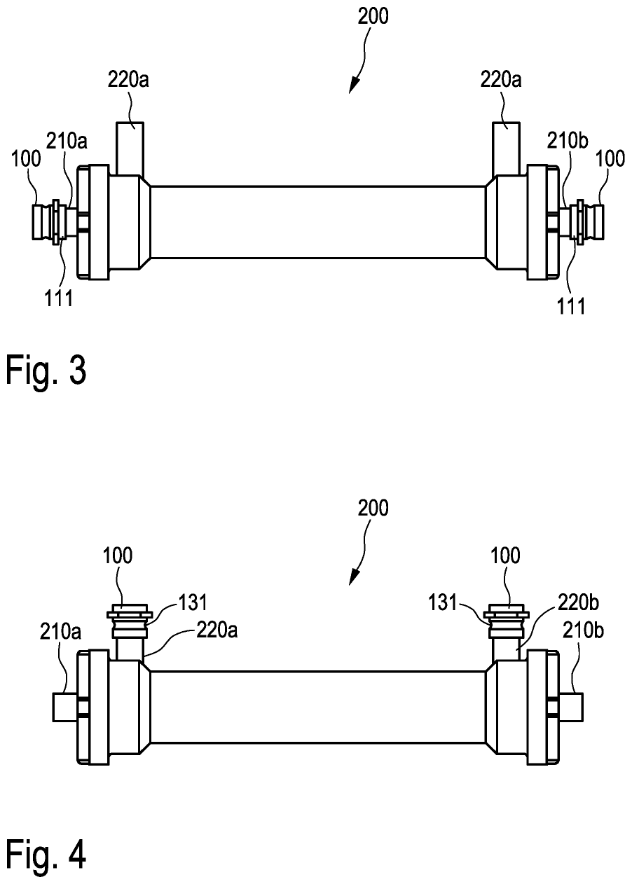

FIG. 3, a schematic representation of a side view of a dialyzer with blood ports closed with closure caps according to the invention,

FIG. 4, a schematic representation of a side view of a dialyzer with dialysate port closed with closure caps according to the invention,

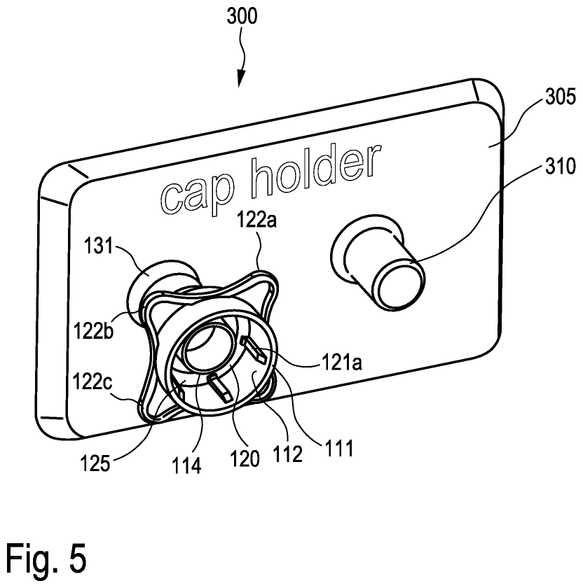

FIG. 5, a schematic representation of a closure cap holder.

### DETAILED DESCRIPTION OF THE INVENTION

In a first aspect, the invention relates to a closure cap 100 for closing two different fluid ports of a dialyzer, wherein a first fluid port of the dialyzer has at least a first circumferential outer surface and optionally a second circumferential outer surface surrounding the first circumferential outer surface, and a second fluid port of the dialyzer has at least one circumferential outer surface,
the closure cap comprising,
a first receiving portion 110 for receiving the one first fluid port and a second receiving portion 130 for receiving the one second fluid port, and further a component body 150 connecting the first receiving portion to the second receiving portion,
the first receiving portion comprising,
a first circumferential side wall 111 having an inner side 112 concave to the first receiving portion, comprising at least one circumferential inner surface 113,
a sleeve 114 disposed radially of the first circumferential side wall, having a convex shaped outer side 115 having at least one circumferential outer surface 116, and a concave shaped inner side 117 having at least one circumferential inner surface 118,
wherein the first circumferential side wall 111 and the radially disposed sleeve 114 adjoin the component body 150, and wherein the at least one circumferential outer side 115 of the radially disposed sleeve 114 and the at least one circumferential inner side 112 of the first circumferential side wall face each other and are spaced apart and form an annular gap 119,
wherein the first circumferential side wall 111 adjoining the component body 150 extends from the component body 150 in the direction of a first open end side 125 of the first receiving portion 110 and defines the first receiving portion 110, and the radially disposed sleeve 114 adjoining the component body 150 extends from the component body 150 in the direction of the one first open end side 125,
further wherein the at least one circumferential inner surface 118 of the radially disposed sleeve 114 is configured to cooperate with the at least one circumferential outer surface of the first fluid port of the dialyzer in a liquid-tight manner,
the second receiving portion 130 comprising,
a second circumferential side wall 131 having an inner side 132 concave to the second receiving portion 130 and having at least one circumferential inner surface 133, the second side wall 131 adjoining the component body 150 and extending from the component body 150 toward a second open end face 140 of the second receiving portion 130 and bounding the second receiving portion 130, further wherein the at least one circumferential inner surface 133 of the second circumferential side wall 131 is configured to cooperate with the circumferential outer surface of the second fluid port of the dialyzer in a liquid-tight manner,
the component body comprising,
at least one liquid-tight end section,
characterized in that the first receiving portion and the second receiving portion are separated from each other by the component body.

In the context of the present invention, a “dialyzer” is understood to mean a hollow fiber membrane filter for extracorporeal blood treatment that has at least two fluid ports for blood and at least one other fluid port for dialysate or filtrate. In the context of the present invention, the term dialyzer refers to such hollow fiber membrane filters used in extracorporeal blood treatment. In particular, in the context of the present application, the term “dialyzer” is understood to mean hollow fiber membrane filters used in dialysis, hemodiafiltration, hemofiltration, plasma separation or blood oxygenation as blood treatment filters.

The term “two different fluid ports” means in the context of the present application that two fluid ports are different from each other with respect to their geometry. That means, that the fluid ports may have different inner diameters, outer diameters, surfaces, chamfers, protrusions, and recesses, and the two different fluid ports are different in at least one of these aspects.

In the context of the present application, “circumferential outer surface” is understood to mean a surface extending on the circumference of a component and enclosing the component. “Circumferential” in this context means that a line can be mapped onto the surface, go around the surface without interruption.

In the context of the present application, “circumferential inner surface” is understood to mean a circumferential surface of a component enclosing a cavity. “Circumferential” in this context means that a line can be mapped on the surface that goes around the surface without interruption.

Within the context of the present application, the term “closure cap” is understood to mean a cover which closes an opening in a liquid-tight manner. For the purposes of the present application, “liquid-tight” is understood to mean that within usual application no liquid can pass through a component of the closure cap or escape through an opening closed by the closure cap.

Within the context of the present application, the term “receiving portion” refers to a portion of the closure cap into which a first or second fluid port is insertable.

Within the present application, the term “circumferential sidewall” means a sidewall that extends along a circumference. Within that meaning, a line can be mapped on a circumferential sidewall, which is not interrupted.

Within the present application, the term “component body” is understood to mean a structural section of the closure cap to which a functionality is to be attributed. In particular, the component body is sealing and structurally connecting the first and second sidewalls of the closure cap.

Within the present application, the term “concave shaped inner side” is understood to mean a curved component whose surface points can be connected with a line without this line passing through the component itself. A “concave shaped inner side” thus forms a cavity.

Within the present application, the term “convex shaped outer side” is understood to mean a curved component, wherein surface points of the component can be connected with a line and this line extends completely through the component.

Within the present application, the term “sleeve” is understood to mean a tubular component which defines a cavity and is open on at least one end face of the sleeve. The outer surface of a sleeve as defined in the present application may describe the shape of a lateral surface of a cylinder. Similarly, the inner surface of this sleeve may have the shape of a lateral surface of a cylinder. Deviating from this, the outer surface of the sleeve may describe the lateral surface of a truncated cone and the inner surface of the sleeve may describe the lateral surface of a cylinder and vice versa.

The term “spaced” means enclosing a space.

The term “annular gap” means a gap between two components that forms a ring in its geometry. Within the present application, the term “annular gap” further means that a line encircling the slit can be mapped in the slit that passes through the slit without interruption. In an alternative, the term means that a circular line encircling the slit can be mapped in the slit.

The term “radially disposed sleeve” is understood to mean a sleeve as defined above that is surrounded by the circumferential first side wall so that the sum of all distances in all directions to the circumferential side wall assumes the smallest possible value. “Radial” in this context means “directed towards the center”, where center means the center of gravity of the area circumscribed by the first sidewall. Where the first sidewall is substantially of cylindrical shape, the area circumscribed by the first sidewall is a circle and the center thereof is the center of the circle.

Within the present application, the area circumscribed by the first sidewall as defined above is synonymous with the “first open end side” of the closure cap.

Similarly, within the present application, the area circumscribed by the second side wall is synonymous with the “second open end face” of the closure cap.

For the purposes of the present application, the term “cooperating in a liquid-tight manner” means that a surface of the closure cap and a surface of the fluid ports are in flat contact with each other when pressure is applied so that no liquid can penetrate the surfaces in contact with each other. The pressure is caused by a force-fit and/or form-fit insertion of the fluid port in the receiving portion.

Within the present application, the term “liquid-tight end section” means a part of the component body which bounds the first receiving portion and/or the second receiving portion to the rear of the respective open first or second end face of the closure cap, in particular also bounds it in such a way that no liquid can pass from the first receiving portion into the second receiving portion.

The previously described embodiment is illustrated with reference to FIG. 1. In the embodiment shown, the first receiving portion 110 has a larger inner diameter than the second receiving portion 130. In addition, the radially disposed sleeve 114 is worked out shorter in the direction of extension from the component body 150 to the open first end face 125 than the circumferential first side wall.

According to the illustration in FIG. 1, the inner diameter of the radially disposed sleeve 114 is smaller than the inner diameter of the second receiving portion 130. In particular, the depth of the first receiving portion 110 in the direction of extension from the component 150 to the open first end face 125 is also greater than the depth of the second receiving portion 130 in the direction of extension from the component body 150 to the open second end face 140.

In another embodiment according to the first aspect, the closure cap 100 is characterized in that the at least one circumferential inner surface 113 of the first side wall 111 and/or the at least one circumferential inner surface 133 of the second side wall 131 and/or the at least one circumferential outer surface 116 of the radially disposed sleeve 114 and/or the at least one circumferential inner surface 118 of the radially arranged sleeve 114, have the lateral surface of a cylinder or a truncated cone.

According to FIG. 1, the circumferential inner surface 113 of the first circumferential side wall 111, the circumferential inner surface 133 of the second circumferential side wall 131, and the circumferential inner surface 118 of the radially disposed sleeve have the lateral surface of a cylinder. In this embodiment, the circumferential outer surface 116 of the radially disposed sleeve 114 describes, in one portion, the lateral surface of a truncated cone. In particular, the wall cross-section of the radially disposed sleeve tapers in the direction of the first open end face 125 and can thus be designed to be flexible in this section. This results in an improved fluid seal of the inner surface 118 of the radially disposed sleeve 114 with the circumferential outer surface of a first fluid port, particularly a blood port of a dialyzer. Alternatively, the circumferential inner surface 113, the circumferential inner surface 133, the circumferential inner surface of the 118 may be in shape of a truncated cone to provide an improved fluid seal and/or an improved frictional connection of the closure cap to that of a first circumferential outer surface of a first fluid port, particularly a blood port, of a dialyzer.

In another embodiment according to the first aspect, the closure cap 100 is characterized in that the first circumferential side wall 111 and/or the second circumferential side wall 131 are substantially circular cylindrical. The term “substantially circular-cylindrical” in this context means that the first and second sidewalls describe a circle in a cross-section. “Substantially” means that the side walls 111 and 131 may have protrusions, such as ribs 121 a-c, or may have recesses without disturbing the circular cross section. FIG. 1 shows an embodiment in which the first and second side walls 111 and 131 are arranged rotationally symmetrical about an extension axis A and adopt the defined cylindrical shape.

In another embodiment according to the first aspect, the closure cap 100 is characterized in that the first side wall 111, the radially disposed sleeve 114 and the second side wall 131, extend from the component body in opposite directions to the respective first and second open end faces 125, 140, in particular extending along a common longitudinal axis A from each other in opposite directions. The corresponding embodiment shown in FIG. 1 describes a geometry of the closure cap that can be produced in a single-stage injection molding process. As a result, the production of the closure cap requires little manufacturing effort and is thus inexpensive to produce.

In another embodiment according to the first aspect, the closure cap 100 is characterized in that the end section of the component body 150 comprises or consists of an end wall 152 and the first side wall 111 and/or the second side wall 131 extend(s) perpendicularly from the end wall in the respective directions of the first and second open end faces 125, 140. The corresponding embodiment shown in FIG. 1 shows the end wall 152 as part of the component body 150, wherein a first surface 153 of the end wall faces the first receiving portion 110 and the open first end face 125, and a second surface 154 of the end wall faces the second receiving portion 130 and the open second end face 140. In this embodiment, the end wall 152 separates the first receiving portion 110 from the second receiving portion 130 in a liquid-tight manner. The first and second surfaces 153 and 154 of the end wall 152 may also respectively serve as stop surfaces for the first and second fluid ports once they are inserted into the respective first and second receiving portions 110, 130.

In another embodiment according to the first aspect, the closure cap 100 is characterized in that the component body 150 has, in the annular gap 119 between the inner side 112 of the first circumferential side wall 111 and the outer side 115 of the radially arranged sleeve 114, a circumferential stop surface 120 that annularly surrounds the inner side 112 of the first circumferential side wall 111. The stop surface thus described serves to define the depth of penetration of the first fluid port of a dialyzer, in particular the blood port, into the first receiving portion 110. In this regard, the circumferential stop surface 120 is geometrically configured to abut the upper edge of a circumferential sleeve, which may be part of the first fluid port, in particular the blood port of the dialyzer with the closure cap 100 when connecting the first fluid port, in particular the blood port, in particular a blood port defined according to DIN EN ISO 8637: March 2014 in accordance with section 4.4.3 of the dialyzer with the closure cap 100, and to define the depth of insertion of the fluid port into the first receiving portion 110.

In another embodiment according to the first aspect, the closure cap 100 is characterized in that protruding ribs 121a, 121b, 121c are arranged on the inner side 112 of the first side wall 111. The corresponding embodiment shown in FIG. 1 shows three of these protruding ribs 121a, 121b, 121c in a cross-sectional view. A total of 12 or more or 6 or 8 ribs may be circumferentially arranged on the inner side 112 of the side wall 111. The ribs serve to create a press fit of the closure cap on the first fluid port, particularly the blood port, of the dialyzer. The ribs are thereby pressed onto the circumferential outer surface of the first fluid port, in particular the blood port, of the dialyzer when the closure cap is pushed onto the first fluid port, in particular the blood port, in particular a blood port according to DIN EN ISO 8637: March 2014. This results in a force-fit interaction of the closure cap 100 and the fluid port, which is sufficient to connect the closure cap and fluid port in a liquid-tight manner. In a further embodiment according to the first aspect, the ribs (121a, 121b, 121c) are configured to cooperate with the first fluid port of the dialyzer in a force-fit and/or form fit manner.

As shown in FIG. 1, the ribs 121a, 121b, 121c may be spaced from each other at uniform angular intervals on the inner side 112 of the first circumferential side wall 111.

In principle, the embodiments described so far above may be applicable to fluid ports that are not described by standards. In particular, the closure cap 100 is also intended for fluid ports of a dialyzer that are standardized according to DIN EN ISO 8637: March 2014.

In another embodiment according to the first aspect, the closure cap 100 is characterized in that the at least one circumferential surface 118 on the inner side 117 of the radially disposed sleeve 114 is configured to form a liquid-tight connection with the one first circumferential outer surface of the first fluid port according to DIN EN ISO 8637: March 2014. The first fluid port of a dialyzer according to DIN EN ISO 8637: March 2014 is in particular a blood port. According to DIN EN ISO 8637: March 2014, the first circumferential outer surface of the blood port is located on the male connection part of the blood port. In particular, in this embodiment, the inner side 117 of the radially disposed sleeve 114 may be tapered to cooperate with a tapered embodiment of the male connector portion of the blood port according to DIN EN ISO 8637: March 2014. The upper edge of the male connection portion of the blood port according to DIN EN ISO 8637: March 2014 may abut the first surface 153 of the end plate 152 in a liquid-tight position consisting of the closure cap and the fluid port, while at the same time the upper edge of the threaded collar of the blood port according to DIN EN ISO 8637: March 2014 abuts the circumferential stop surface 120. In an alternative embodiment, the insertion depth of the first fluid port according to DIN EN ISO 8637: March 2014 is defined solely by the threaded collar of the blood port according to DIN EN ISO 8637: March 2014 and the circumferential stop surface 120.

In a further embodiment according to the first aspect, the closure cap 100 is characterized in that the at least one circumferential surface 133 of the inner side 132 of the second circumferential side wall 131 is configured to form a liquid-tight connection with a circumferential outer surface of the second fluid port according to DIN EN ISO 8637: March 2014. In particular, the second fluid port of a dialyzer according to DIN EN ISO 8637: March 2014 is a dialysate port as described in section 4.4.5 of the standard. In one embodiment, the diameter of the second receiving portion 130 is dimensioned such that the closure cap is fixed on the dialysate port in a liquid-tight and force-fit manner between the second receiving portion and the dialysate port. In alternative embodiments, additional locking means may be provided in or on the second receiving portion to provide an additional force-fit and/or form-fit connection between the closure cap 100 and the dialysate port according to DIN EN ISO 8637: March 2014. Screw, bayonet or snap connections can be used as locking means.

In another embodiment according to the first aspect, the closure cap 100 is characterized in that the respective inner ide 112, 132 of the first circumferential sidewall 111 and/or the second sidewall 131 are formed without threads or wherein the respective inner side 112, 132 of the first sidewall 111 and/or the second sidewall 131 do not comprise means to engage a thread. According to this embodiment, it may be provided that both the first receiving portion 110 and the second receiving portion 130 are connectable to the first fluid port or the second fluid port, respectively, in a liquid-tight manner solely via a force-fit connection. Alternatively, in certain embodiments, even if the closure cap 100 is formed without threads and also has no means to engage into a thread, it is not excluded that further locking means are present on the closure cap to assist the connection between the closure cap 100 and the first or second fluid port of the dialyzer.

In another embodiment according to the first aspect, the closure cap 100 is characterized in that the first and second sidewalls 111, 131, the radially disposed sleeve 114, and the component body 150 form an integral component. The term “integral” in this context means that the first and second side walls 111, 131, the radially disposed sleeve 114 can be separated from each other only with irreversible destruction of the component. In particular, this definition does not exclude a multi-component structure. In certain embodiments, first and second sidewalls 111, 131, sleeve 114, and component body 150 may comprise individual components joined together by bonding, welding, or multi-component injection molding.

In another embodiment according to the first aspect, the closure cap 100 is characterized in that the closure cap 100 is one-piece. In this context, the term “one-piece” means that the closure cap 100 comprises only one material component and the closure cap has no interfaces within that material component. Such an embodiment is shown schematically in FIG. 1. This embodiment has the advantage that the closure cap can be manufactured in a single-stage injection molding process.

In a further embodiment according to the first aspect, the closure cap 100 is characterized in that the closure cap is formed flexible, at least in sections. “Flexibility” of the portions of the closure cap 100 can be achieved by an appropriate selection of polymers from which the closure cap is made. In particular, the closure cap 100 may comprise a blend of different polymers. In certain embodiments, the closure cap 100 is configured to be flexible in sections by having a portion of an elastomeric polymer admixed with the closure cap material. Preferred closure cap materials are selected from the group comprising polyvinyl chloride, polyolefin, polyester, polyamide, polysulfone, polyetherimide, polyether sulfone, polyphenylene sulfide, polyether ketone, polyether ether ketone, ABS resin, polystyrene, polybutadiene, polyacrylate, polyacrylonitrile, polyacetal, polycarbonate, polyphenylene ether, ethylene-vinyl acetate copolymer, polyvinyl acetate, liquid crystal polymer, ethylene-tetrafluoroethylene copolymer, aromatic polyester, polyvinyl fluoride, polyvinylidene fluoride, polyvinylidene chloride, and mixtures thereof.

In alternative embodiments, sections of the closure cap 100 may be formed with a low wall thickness such that a provided flexibility of the closure cap in these sections is achieved. A predetermined flexibility of portions of the closure cap allows the closure cap to be placed on a respective first or second fluid port of the dialyzer under material strain and pre-tensioning. This increases the force-fit closure of the connection between the closure cap and the respective first or second fluid port.

In another embodiment of the first aspect, the closure cap is characterized in that closure cap comprises handle elements 122a, 122b, 122c. The grip elements are preferably provided on the outer side of the closure cap, in particular on the outer side 123 of the first side wall 111, the outer side 155 of the component body 150 or the outer side of the second side wall 134. According to FIG. 2, the handle elements 122a, 122b, 122c are preferably designed as protruding wing elements. They facilitate the application and removal of the cap from a respective first or second fluid port. The number of handle elements can be 4 or 6 or 8 or 10 or 12.

In a second aspect, the invention relates to a dialyzer 200 comprising at least two first fluid ports for blood 210a, 210b, hereinafter also referred to as blood ports, and at least one second fluid port for dialysate or filtrate 220a, 220b, hereinafter also referred to as dialysate port, wherein the at least two first fluid ports 210a, 210b cooperate and are closed with the first receiving portion 110 of a closure cap 100 according to one of the embodiments of the first aspect of the invention as described above. A such embodiment is shown schematically in FIG. 3. In this figure, the fluid ports for dialysate and blood are shown schematically. According to FIG. 3, the sealing cap and the blood port are shown in the connection state. The blood port is inserted in the first receiving portion 110 of the closure cap 100. The first sidewall encloses the blood ports 210a, 210b. In the case of a blood port according to DIN EN ISO 8637: March 2014, according to FIG. 3, the first receiving portion 110 of the closure cap encloses the threaded collar of the blood port.

In a third aspect, the invention relates to a dialyzer 200 comprising at least two first fluid ports for blood 210a, 210b, i.e. blood ports, and at least one second fluid port for dialysate or filtrate 220a, 220b, i.e. dialysate ports, wherein the at least one second fluid port 220a, 220b cooperates with and is sealed to the second receiving portion 130 of a closure cap 100 according to one of the embodiments of the first aspect of the invention described above. Such an embodiment is schematically shown in FIG. 4. In this figure, the fluid ports for dialysate and blood are shown schematically only. The dialysate ports according to FIG. 4 are closed with the closure caps 100. In this case, the dialysate port is inserted into the second receiving portion 130 (not shown in FIG. 3) of the closure cap 100. According to the embodiment shown in FIG. 4, the second side wall 131 encloses the dialysate port. In the case of a dialysate port according to DIN EN ISO 8637: March 2014, according to FIG. 4, the second receiving portion 130 of the closure cap 100 encloses the circumferential cylindrical outer surface of the dialysate port.

In a fourth aspect, the invention relates to a closure cap holder 300, for receiving a closure cap 100 according to one of the embodiments according to the first aspect of the invention, comprising at least one holder 310 that can cooperate with the first and/or the second receiving portion 110, 130 of the closure cap 100 and can fix the closure cap. Such an embodiment is shown in FIG. 5. FIG. 5 shows a closure cap holder 300 having a holder 310 and a closure cap 100 mounted on another holder. The holder is mounted on a structural plate 305. According to FIG. 5, the closure cap 100 is fixed by a holder via the second receiving portion 130. This holder in FIG. 5 is covered by the second side wall 131 of the closure cap 100 but corresponds in geometry to the holder 310. The holder is overlaid by the first sidewall 131. The open first end face 125 of the closure cap 100 is shown in FIG. 5 facing the viewer. Alternatively, the holder may be configured to cooperate with the annular gap 119 of the closure cap and to fix the closure cap, e.g. via force-fit or form-fit locking. In another alternative embodiment, the holder 310 may also be configured to cooperate with the inner side 117 of the radially disposed sleeve 114 and to fix the closure cap, e.g. via force-fit or form-fit locking.

In one embodiment of the fourth aspect, the closure cap holder 300 comprises the structural plate 305, wherein the structural plate comprising the at least one holder 310 is formed as a cylindrical, conical, or frustoconical protrusion, and/or wherein the holder 310 or a further holder is formed as a recess in the structural plate, and the recess is geometrically configured to cooperate with the first receiving portion 110 or the second receiving portion 130 of the closure cap 100 according to an embodiment of the first aspect of the invention in a force-fit and/or form-fit locking manner.

A fifth aspect of the invention relates to a dialysis machine comprising a cap holder according to one of the embodiments of the fourth aspect of the invention.

1. A closure cap for closing two different fluid ports of a dialyzer, wherein a first fluid port of the dialyzer has at least a first circumferential outer surface and optionally a second circumferential outer surface surrounding the first circumferential outer surface, and a second fluid port of the dialyzer has at least one circumferential outer surface,
the closure cap comprising,
a first receiving portion for receiving the one first fluid port and a second receiving portion for receiving the one second fluid port, and further a component body connecting the first receiving portion to the second receiving portion,
the first receiving portion comprising,
a first circumferential side wall having an inner side concave to the first receiving portion, comprising at least one circumferential inner surface,
a sleeve disposed radially of the first circumferential side wall, having a convex shaped outer side having at least one circumferential outer surface, and a concave shaped inner side having at least one circumferential inner surface,
wherein the first circumferential side wall and the radially disposed sleeve adjoin the component body, and wherein the at least one circumferential outer side of the radially disposed sleeve and the at least one circumferential inner side of the first circumferential side wall face each other and are spaced apart and form an annular gap,
wherein the first circumferential side wall adjoining the component body extends from the component body in the direction of a first open end face of the first receiving portion and defines the first receiving portion, and the radially disposed sleeve adjoining the component body extends from the component body in the direction of the one first open end face,
further wherein the at least one circumferential inner surface of the radially disposed sleeve is configured to cooperate with the at least one circumferential outer surface of the first fluid port of the dialyzer in a liquid-tight manner,
the second receiving portion comprising,
a second circumferential side wall having an inner side concave to the second receiving portion and having at least one circumferential inner surface, the second side wall adjoining the component body and extending from the component body toward a second open end face of the second receiving portion and bounding the second receiving portion, further wherein the at least one circumferential inner surface of the second circumferential side wall is configured to cooperate with the circumferential outer surface of the second fluid port of the dialyzer in a liquid-tight manner,
the component body comprising,
at least one liquid-tight end section,
wherein the first receiving portion and the second receiving portion are separated from each other by the component body.

2. The closure cap according to claim 1, wherein
the at least one circumferential inner surface of the first side wall and/or
the at least one circumferential inner surface of the second side wall and/or
the at least one circumferential outer surface of the radially disposed sleeve and/or
the at least one circumferential inner surface of the radially disposed sleeve,
have the lateral surface of a cylinder or a truncated cone.

3. The closure cap according to claim 1, wherein
the first circumferential side wall and/or the second circumferential side wall are substantially circular cylindrical.

4. The closure cap according to claim 1, wherein the first side wall, the radially disposed sleeve and the second side wall, extend from the component body in opposite directions to the first and second open end face, respectively, and extend along a common longitudinal axis from each other in opposite directions.

5. The closure cap according to claim 1, wherein the end portion of the component body comprises or consists of an end wall and the first side wall and/or the second side wall extend(s) perpendicularly from the end wall in the respective directions of the first and second open end face.

6. The closure cap according to claim 1, wherein the component body has, in the annular gap between the inner side of the first circumferential side wall and the outer side of the radially arranged sleeve, a circumferential stop surface which annularly surrounds the inner side of the first circumferential side wall.

7. The closure cap according to claim 1, wherein protruding ribs are arranged on the inner side of the first side wall.

8. The closure cap of claim 7, wherein the ribs are spaced at uniform angular intervals on the inner side of the first circumferential side wall.

9. The closure cap according to claim 7, wherein the ribs are configured to cooperate with the first fluid port of the dialyzer in a force-fit and/or form-fit locking manner.

10. The closure cap according to claim 1, wherein the at least one circumferential surface on the inner side of the radially disposed sleeve is configured to form a liquid-tight connection with the one first circumferential outer surface of the first fluid port according to DIN EN ISO 8637: March 2014.

11. The closure cap according to claim 10, wherein the circumferential stop surface defines the insertion depth of the first fluid port into the first receiving area of the closure cap.

12. The closure cap according to claim 1, wherein the at least one circumferential surface of the inner side of the second circumferential side wall is configured to form a liquid-tight connection with the circumferential outer surface of the second fluid port according to DIN EN ISO 8637: March 2014.

13. The closure cap according to claim 1, wherein the respective inner side of the first circumferential side wall and/or the second side wall are formed without threads, or wherein the respective inner side of the first side wall and/or the second side wall have no means to engage a thread.

14. The closure cap according to claim 1, wherein the first and second sidewalls, the radially disposed sleeve, and the component body form an integral component.

15. The closure cap according to claim 1, wherein the closure cap is an integral part.

16. The closure cap according to claim 1, wherein the closure cap is worked out to be flexible at least in sections.

17. The closure cap according to claim 1, wherein the closure cap comprises a polymeric material selected from the group of polyvinyl chloride, polyolefin, polyester, polyamide, polysulfone, polyetherimide, polyether sulfone, polyphenylene sulfide, polyether ketone, polyether ether ketone, ABS resin, polystyrene, polybutadiene, polyacrylate, polyacrylonitrile, polyacetal, polycarbonate, polyphenylene ether, ethylene-vinyl acetate copolymer, polyvinyl acetate, liquid crystal polymer, ethylene-tetrafluoroethylene copolymer, aromatic polyester, polyvinyl fluoride, polyvinylidene fluoride, polyvinylidene chloride, and mixtures thereof.

18. A dialyzer comprising at least two first fluid ports for blood and at least one second fluid port for dialysate or filtrate, wherein the at least two first fluid ports cooperate with and are sealed to the first receiving portion of the closure cap according to claim 1.

19. A dialyzer comprising at least two first fluid ports for blood and at least one second fluid port for dialysate or filtrate, wherein the at least one second fluid port cooperates with and is closed by the second receiving portion of the closure cap according to claim 1.

20. A closure cap holder, for receiving the closure cap according to claim 1, comprising at least one holder cooperating with the first or the second receiving portion of the closure cap.

21. The closure cap holder of claim 20 comprising a structural plate, wherein the structural plate comprises the at least one holder formed as a cylindrical, conical or frustoconical protrusion, and/or wherein the holder is formed as a recess in the structural plate and the recess is geometrically configured to cooperate with the first receiving portion or the second receiving portion of the closure cap in a in a force-fit and/or form-fit locking manner.

22. A dialysis machine comprising the closure cap holder according to claim 20.
